SEQUENCE LISTING (8 sequences)

SEQ ID NO: 1 - DNA, 22 bp, Artificial Sequence
  PCR primer
catctgcgacagtcgagttctg

SEQ ID NO: 2 - DNA, 22 bp, Artificial Sequence
  PCR primer
cacaacccatcgtgacattttc

SEQ ID NO: 3 - DNA, 22 bp, Artificial Sequence
  PCR primer
gaatataatcccaagcgatttg

SEQ ID NO: 4 - DNA, 21 bp, Artificial Sequence
  PCR primer
gtcaagacctttttaccacac

SEQ ID NO: 5 - DNA, 24 bp, Artificial Sequence
  PCR primer
aagcagggcagacattgcgatacc

SEQ ID NO: 6 - DNA, 24 bp, Artificial Sequence
  PCR primer
agatgcagatggggaagttggtgt

SEQ ID NO: 7 - DNA, 21 bp, Artificial Sequence
  PCR primer
caatagtgatgacctggccgt

SEQ ID NO: 8 - DNA, 20 bp, Artificial Sequence
  PCR primer
agagggaaatcgtgcgtgac

(19) United States
(12) Patent Application Publication
(10) Pub. No.: US 2015/0126620 A1
(43) Pub. Date: May 7, 2015

(54) METHODS FOR TREATING ANEMIA

(71) Applicants: Thomas B. Neff, Atherton, CA (US); David Liu, Palo Alto, CA (US); Gail Walkinshaw, Mountain View, CA (US); Marja Johanna Myllyharju, Oulu (FI); Kari Ilkka Kivirikko, Oulu (FI); Peppi Leena Elina Karppinen, Oulu (FI)

(72) Inventors: Thomas B. Neff, Atherton, CA (US); David Liu, Palo Alto, CA (US); Gail Walkinshaw, Mountain View, CA (US); Marja Johanna Myllyharju, Oulu (FI); Kari Ilkka Kivirikko, Oulu (FI); Peppi Leena Elina Karppinen, Oulu (FI)

(73) Assignee: FibroGen, Inc., San Francisco, CA (US)

(21) Appl. No.: 13/587,743

(22) Filed: Aug. 16, 2012

Related U.S. Application Data
(60) Provisional application No. 61/524,715, filed on Aug. 17, 2011.
(60) Provisional application No. 61/670,048, filed on Jul. 10, 2012.

(51) Int. Cl. A61K 45/06

(52) U.S. Cl. CPC A61K 45/06
USPC 514/789

(57) ABSTRACT

The present invention relates to improved methods and compounds for treating anemia. Screening methods to identify agents for use in these treatment methods are also provided.

This application claims the benefit of U.S. Provisional Application Ser. No. 61/524,715, filed on 17 Aug. 2011 and U.S. Provisional Application Ser. No. 61/670,048, filed on 10 Jul. 2012, each of which is incorporated by reference herein in its entirety.

### FIELD OF THE INVENTION

The present invention relates to improved methods for treating anemia and agents that can be used in these methods.

### BACKGROUND OF THE INVENTION

Anemia is any abnormality in red blood cells (erythrocytes) or hemoglobin that reduces the oxygen-carrying capacity of blood. It is the most common blood disorder and is associated with the feeling of weakness or fatigue, dizziness, drowsiness and poor cognition leading to a decreased quality of life. Additionally, subjects with severe cases of anemia have difficulty breathing and may develop heart abnormalities.

The generation of red blood cells is under the control of erythropoietin (EPO). In adults, this hormone is primarily secreted by the kidneys. Under hypoxic conditions, the body compensates for decreased oxygen availability by secreting increased levels of EPO, thereby generating more erythrocytes. The level of EPO expression is under the control of oxygen sensing enzymes termed hypoxia-inducible transcription factor prolyl hydroxylases (HIF PHs).

The role of HIF PHs in the regulation of the response to hypoxia, in general, or the regulation of EPO production and erythropoiesis, in particular, is well defined. Three HIF PH isozymes are found in the cytoplasm (HIF PH 1, HIF PH 2 and HIF PH 3). (For discussion of these HIF PH isozymes see Bruick R K et al. Science. 2001; 294:1337-1340; Epstein A C R et al. Cell. 2001; 107:43-54; Ivan M et al. Proc Natl Acad Sci USA. 2002; 99:13459-13464.) Under normoxic conditions, HIF PHs hydroxylate the a subunit of HIF targeting it for destruction and preventing appreciable accumulation of the HIF heterodimer transcription factor. (See Ivan M et al. Science. 2001; 292:464-468; Jaakkola P et al. Science. 2001; 292:468-472; and Yu F et al. Proc Natl Acad Sci USA. 2001; 98:9630-9635.) Under hypoxic conditions, the activity of HIF PHs is markedly reduced, allowing HIF-α subunits to accumulate and leading to the formation of the HIF heterodimer, and ultimately increasing the transcription rate of EPO.

An additional prolyl hydroxylase enzyme capable of hydroxylating HIF residues has been identified. This enzyme (P4H-TM) resides in the lumen of the endoplasmic reticulum and can be differentiated from the HIF PH enzymes by the possession of a transmembrane domain. P4H-TM has not yet been fully characterized. (For discussion see Oehme F et al. Biochem Biophys Res Commun. 2002; 296:343-349 and Koivunen P et al. J Biol Chem. 2007; 282:30544-30552.) The protein sequence of P4H-TM more closely resembles those of the collagen prolyl 4-hydroxylases (C-P4Hs) than that of the HIF PHs. An in vivo function for the P4H-TM enzyme has not been conclusively demonstrated.

Current treatments for anemia rely on the injection of recombinant EPO, an inconvenient treatment that is associated with increased incidences of cardiovascular complications in significant patient populations. Recombinant EPO also requires refrigeration, limiting its distribution to more developed regions. Small molecule inhibitors of HIF PH have been disclosed for use in treating anemia (See, e.g., PCT/US02/39163 and PCT/US2004/017772) with select molecules currently in clinical trials. The present invention provides improved methods for treating anemia, whereby robust induction of endogenous EPO can be achieved by inhibiting P4H-TM activity alone or in combination with the inhibition of HIF PH activity. Additionally, screening methods to identify agents for use in these treatment methods are also provided.

### SUMMARY OF THE INVENTION

In one aspect of the invention, a method is provided for treating anemia in a subject in need thereof, the method comprising administering to the subject an effective amount of a first agent that inhibits HIF PH activity and an effective amount of a second agent that inhibits P4H-TM activity, thereby treating the anemia. In some embodiments, the first and second agent are the same. In other embodiments, the therapeutic dosing ranges for the first and second agent are substantially the same. In further embodiments, HIF PH is selected from the group consisting of HIF PH 1, HIF PH 2, and HIF PH 3.

In some embodiments, the first agent inhibits HIF PH 1, HIF PH 2 and HIF PH 3 activity. In further embodiments, the therapeutic dosing ranges for the first agent to inhibit HIF PH1, HIF PH 2 and HIF PH3 activity are substantially the same.

In other embodiments, the first agent inhibits HIF PH 1 and HIF PH 2 activity. In further embodiments, the therapeutic dosing ranges for the first agent to inhibit HIF PH1 and HIF PH 2 activity are substantially the same.

In some embodiments, the first agent inhibits HIF PH1 and HIF PH3 activity. In further embodiments, the therapeutic dosing ranges for the first agent to inhibit HIF PH1 and HIF PH 3 activity are substantially the same.

In other embodiments, the first agent inhibits HIF PH2 and HIF PH3 activity. In further embodiments, the therapeutic dosing ranges for the first agent to inhibit HIF PH2 and HIF PH 3 activity are substantially the same.

In another aspect of the invention, a method is provided for treating anemia in a subject in need thereof, the method comprising administering to the subject an effective amount of an agent that inhibits P4H-TM activity, thereby treating the anemia.

In a further aspect of the invention, a method is provided for increasing endogenous erythropoietin in a subject in need thereof, the method comprising administering to the subject an effective amount of a first agent that inhibits HIF PH activity and an effective amount of a second agent that inhibits P4H-TM activity, thereby increasing endogenous erythropoietin.

In another aspect, a method is provided for increasing hematocrit in a subject in need thereof, the method comprising administering to the subject an effective amount of a first agent that inhibits HIF PH activity and an effective amount of a second agent that inhibits P4H-TM activity, thereby increasing hematocrit.

In one aspect, a method is provided for increasing hemoglobin in a subject in need thereof, the method comprising administering to the subject an effective amount of a first agent that inhibits HIF PH activity and an effective amount of a second agent that inhibits P4H-TM activity, thereby increasing hemoglobin.

In another aspect, a method is provided for increasing reticulocytes in a subject in need thereof, the method comprising administering to the subject an effective amount of a first agent that inhibits HIF PH activity and an effective amount of a second agent that inhibits P4H-TM activity, thereby increasing reticulocytes.

In a further aspect, a method is provided for increasing erythrocytes in a subject in need thereof, the method comprising administering to the subject an effective amount of a first agent that inhibits HIF PH activity and an effective amount of a second agent that inhibits P4H-TM activity, thereby increasing erythrocytes.

In another aspect, a method is provided for decreasing hepcidin in a subject in need thereof, the method comprising administering to the subject an effective amount of a first agent that inhibits HIF PH activity and an effective amount of a second agent that inhibits P4H-TM activity, thereby decreasing hepcidin.

### BRIEF DESCRIPTION OF THE DRAWINGS

FIG. 1 are Western blots that illustrate the level of HIF-1α and HIF-2α stabilization in the kidney and liver of vehicle treated and compound A treated wild-type and P4h-tm^{-/-} mice. The compound A treated mice received three oral doses (100 mg/kg) per week for 5 weeks and were sacrificed 6 h after the last dose. HIF-la samples are supernatants from tissue homogenates, while HIF-2α samples are nuclear fractions. The lanes in each image were grouped from different parts of the same gel with the same exposure. α-tubulin (α-tub) and β-actin served as controls. ns=nonspecific.

FIG. 2 are Western blots that illustrate the level of HIF-1α and HIF-2α stabilization in the kidney and liver of vehicle treated and compound A treated wild-type and Hif-ph2^{gt/gt} mice. The compound A treated mice received three oral doses (100 mg/kg) per week for 3 weeks and were sacrificed 6 h after the last dose. HIF-1α samples are supernatants from tissue homogenates, while HIF-2α samples are nuclear fractions. The lanes in each image were grouped from different parts of the same gel with the same exposure. α-tubulin (α-tub) and β-actin served as controls. ns=nonspecific.

FIGS. 3A and 3B compare, respectively, the level of EPO mRNA in the kidney and liver of (3A) vehicle treated and compound A treated wild-type and P4h-tm^{-/-} mice; and (3B) vehicle treated and compound A treated wild-type and Hif-ph2^{gt/gt} mice. The compound A treated mice received three oral doses (100 mg/kg) per week for 5 weeks in P4h-tm^{-/-} and 3 weeks in Hif-ph2^{gt/gt} mice. The animals were sacrificed 6 h after the last dose. EPO mRNA levels are expressed as a percentage of the compound A treated wild-type EPO mRNA level, the means of the latter being taken as 100%. Statistical significance is shown only for comparisons between compound A-treated gene-modified and wild-type mice. * P<05, and *** P=0.001. For mice in 3A, n=5 for compound A-treated mice at 3 weeks, n=13 for the compound A-treated wild-type mice and n=6 for the compound A-treated P4h-tm^{-/-} mice at 5 weeks. For mice in 3B, n=6-8 for all groups. Error bars represent SEM.

FIGS. 4A and 4B compare, respectively, the effect of a single dose of compound A (100 mg/kg) on serum EPO concentrations of (4A) wild-type mice and P4h-tm^{-/-} mice; and (4B) wild-type mice and Hif-ph2^{gt/gt} mice. Vehicle treated mice served as controls. Blood was drawn 6 h after administration and serum EPO concentrations were analyzed. The values for the vehicle-treated wild-type and gene-modified mice were less than 3% of those for the compound A-treated wild-type mice. Statistical significance is shown only for the comparison of the values between compound A-treated gene modified mice and wild-type mice. ** P<0.005 for the difference between the values for the compound A-treated gene-modified mice and wild-type mice. In 4A, n=3 for the two vehicle-treated groups, n=7 for the compound A-treated wild-type mice and n=5 for the compound A-treated P4h-tm^{-/-} mice. In 4B, n=3 for each group. Error bars represent SEM.

FIGS. 5A and 5B compare, respectively, the effect of repeated administration of compound A on serum EPO concentration of (5A) wild-type mice and P4h-tm^{-/-} mice; and (5B) wild-type mice and Hif-ph2^{gt/gt} mice. The compound A-treated mice received three oral doses (100 mg/kg) per week for a total of 3 or 5 weeks. The animals were sacrificed 6 h after the last dose. Vehicle treated mice served as controls. Blood was drawn prior to sacrifice and the EPO level determined Serum EPO levels are expressed as a percentage of the wild-type serum EPO level, the means of the latter being taken as 100%. There were no significant differences between values for vehicle-treated wild-type and gene-modified mice, however, the difference in values for compound A-treated groups were highly significant. Values of n as in FIG. 3. Error bars represent SEM.

FIGS. 6A and 6B compare, respectively, (6A) the hemoglobin content, hematocrit and percentage of reticulocytes following administration of compound A from wild-type mice and P4h-tm^{-/-} mice; and (6B) the hemoglobin content and hematocrit following administration of compound A from wild-type and Hif-ph2^{gt/gt} mice. The compound A treated mice received three oral doses (100 mg/kg) per week for 3-5 weeks and were sacrificed 6 h after the last dose. Vehicle treated mice served as controls. Blood was drawn prior to sacrifice and the EPO level determined. The difference between reticulocyte values for compound A-treated and vehicle-treated mice were significant at 3 weeks in 6A. The differences between hemoglobin and hematocrit values for compound A-treated and vehicle-treated mice were highly significant or significant in 6B. Statistical significance is shown only for the comparison of the values between the compound A-treated gene-modified and compound A-treated wild-type mice. * P<0.02. ** P<0.005. Values of n as in FIG. 3. Error bars represent SEM.

FIGS. 7A and 7B compare, respectively, the effect of compound A treatment on hepatic hepcidin mRNA level in (7A) wild-type mice and P4h-tm^{-/-} mice; and (7B) wild-type mice and Hif-p4h-2^{gt/gt} mice. Mice received three oral doses of compound A (100 mg/kg) per week for 3-5 weeks and were sacrificed 6 h after the last dose. Vehicle treated mice served as controls. Hepcidin mRNA levels are expressed as a percentage of the vehicle-treated wild-type mice hepcidin mRNA level, the means of the latter being taken as 100%. Statistical significance is shown for comparisons between the values for the vehicle-treated and compound A-treated wild-type mice. Statistical significance is also shown for compound A-treated gene-modified and compound A-treated wild-type mice. * P<0.05, *** P<0.0001. There was no significant differences between the values for the vehicle-treated wild-type and vehicle-treated gene-modified mice in 7A and 7B. Values of n as in FIG. 3. Error bars represent SEM.

FIGS. 8A and 8B illustrate, respectively, the change in (8A) kidney EPO mRNA levels and (8B) serum EPO values in Hif-p4h-2^{-/gt}/P4h-tm^{-/-} and Hif-ph2^{gt/gt}/P4h-tm^{-/-} mice compared to controls. Control mice included wild-type, Hif-ph2^{+/+}/P4h-tm^{+/-}, Hif-ph-2^{+/gt}/P4h-tm^{+/+} and Hif-ph2^{+/gt/}/P4h-tm^{+/-} mice. The decrease in serum EPO levels for Hif-ph2^{gt/gt}/P4h-tm^{-/-} mice is statistical significant compared to control mice. * P<0.05. n=21 for controls and n=6 for Hif-ph2^{+/gt}/P4h-tm^{-/-} mice and Hif-ph2^{gt/gt/}P4h-tm^{-/-} mice. Error bars represent SEM.

FIGS. 9A and 9B illustrate, respectively, the change in (9A) hemoglobin and (9B) hematocrit values in Hif-p4h-2^{-/gt}/P4h-tm^{-/-} and Hif-ph2^{gt/gt}/P4h-tm^{-/-} mice compared to controls. Control mice included wild-type, Hif-ph2^{+/+}/P4h-tm^{+/-}, Hif-ph2^{+/gt}/P4h-tm^{+/+} and Hif-ph2^{+/gt}/P4h-tm^{+/-} mice. The increase in hemoglobin and hematocrit values for Hif-ph2^{gt/gt}/P4h-tm^{-/-} mice are statistical significant compared to control mice. *** P<0.00005. n=21 for controls, n=6 for Hif-ph2^{+/gt}/P4h-tm^{-/-} mice and Hif-ph2^{gt/gt/}P4h-tm^{-/-} mice. Error bars represent SEM.

### DESCRIPTION OF THE INVENTION

The terms “HIF prolyl hydroxylase” and “HIF PH” refer to any enzyme without a transmembrane domain that is capable of hydroxylating a proline residue in a HIF protein. Preferably, the proline residue hydroxylated by HIF PH includes the proline found within the motif LXXLAP, e.g., as occurs in the human HIF-1α native sequence at L₃₉₇TLLAP and L₅₅₉EMLAP. HIF PH includes members of the Egl-Nine (EGLN) gene family described by Taylor (Gene. 2001; 275:125-132), and characterized by Aravind and Koonin (Genome Biol 2:RESEARCH0007. 2001), Epstein et al. (supra), and Bruick et al. (supra). Examples of HIF PH enzymes include human SM-20 (EGLN1, HIF PH 2) (GenBank Accession No. AAG33965; Dupuy et al. Genomics. 2000; 69:348-54), EGLN2 isoform 1 (PH 1) (GenBank Accession No. CAC42510; Taylor, supra), EGLN2 isoform 2 (PH 1) (GenBank Accession No. NP_{—}060025), and EGLN3 (HIF PH 3) (GenBank Accession No. CAC42511; Taylor, supra). HIF PH further includes mouse EGLN1 (GenBank Accession No. CAC42515), EGLN2 (GenBank Accession No. CAC42511), and EGLN3 (SM-20) (GenBank Accession No. CAC42517); and rat SM-20 (GenBank Accession No. AAA19321). Additionally, HIF PH may include Caenorhabditis elegans EGL-9 (GenBank Accession No. AAD56365) and Drosophila melanogaster CG1114 gene product (GenBank Accession No. AAF52050). HIF PH also includes any fragment of the foregoing full-length proteins that retain at least one structural or functional characteristic.

The term “HIF P4H-TM” refers to any enzyme possessing a transmembrane domain that is capable of hydroxylating a proline residue in a HIF protein. HIF P4H-TMs include enzymes that have an endoplasmic reticulum transmembrane domain. See e.g., Koivunen P et al. J Bio Chem. 2007; 82(42):30544-30552. In some embodiments, HIF P4H-TM is human (Genbank Accession No. BC011710).

The terms “treating,” “treatment” and the like, are used herein to mean administering a therapy to a patient in need thereof.

A “therapeutically effective dose” of an agent refers to the dose sufficient to effect beneficial or desired results. In some embodiments, the therapeutically effective dose of the agent is the dose sufficient to increase hemoglobin, hematocrit, reticulocytes, erythrocytes or EPO levels in a subject. In other embodiments, the therapeutically effective dose of an agent is the dose sufficient to decrease hepcidin. In further embodiments, the therapeutically effective dose of an agent is the dose sufficient to treat anemia.

It is expected that an agent that inhibits HIF PH and P4H-TM activity will generate a larger induction of EPO expression, a larger increase in serum EPO, a larger increase in hemoglobin, a larger increase in hematocrit, a larger increase in reticulocytes, or a larger decrease in hepcidin compared to the use of an agent that only substantially inhibits HIF PH activity. In some embodiments, the agent inhibits pan HIF PH 1-3 activity and P4H-TM activity. In other embodiments, the agent inhibits HIF PH 1 activity, HIF PH 2 activity and P4H-TM activity without substantially inhibiting HIF PH 3 activity. In additional embodiments, the agent inhibits HIF PH 1 activity, HIF PH 3 activity and P4H-TM activity without substantially inhibiting HIF PH 2 activity. In further embodiments, the agent inhibits HIF PH 2 activity, HIF PH 3 activity and P4H-TM activity without substantially inhibiting HIF PH 1 activity.

It is further expected that the combined use a first agent that inhibits HIF PH and a second agent that inhibits P4H-TM activity will generate a larger induction of EPO expression, a larger increase in serum EPO, a larger increase in hemoglobin, a larger increase in hematocrit, a larger increase in reticulocytes, or a larger decrease in hepcidin compared to the use of the first agent alone.

### Subjects

Subjects suitable for the methods of the invention include any mammal, such as but not limited to, human, non-human primate, sheep, horse, cattle, goat, pig, dog, cat, rat, and mouse. Preferably the subject is a human. Suitable subjects have anemia or are non-anemic and in need of an increase in hemoglobin, hematocrit, reticulocyte or erythrocyte level. The anemia may be associated with or result from a number of other conditions or disorders including but not limited to, chronic kidney disease, dialysis, cancer, chemotherapy and inflammation. Anemic subjects have a lower than normal hemoglobin, hematocrit, reticulocyte and/or erythrocyte level prior to treatment in the method of the invention. Normal hemoglobin levels for various mammalian species are well known in the art. In particular, for humans, normal hemoglobin levels range from 13 g/dL-18 g/dL for males and 12 g/dL-16 g/dL for females. A human subject having mild to moderate anemia will typically have a hemoglobin level of between 10-12 g/dL, typically between 10-11 g/dL, prior to treatment in the method of the invention. Severely anemic subjects can have hemoglobin levels below 10 g/dL, below 8 g/dL, or below 6 g/dL. Normal Hematocrit levels are about 38-47 in non-pregnant females and about 42-54 in males. Hematocrit levels below these lower limits are indicative of the presence of anemia.

A male human subject in need of an increase in hemoglobin level typically has a hemoglobin level lower than 13 g/dL while a female human subject typically has a hemoglobin level lower than 12 g/dL for. Subjects benefiting from the method of the invention include those subjects in need of a rapid increase in hemoglobin level such as subjects having very low hemoglobin levels, e.g., severely anemic patients. A “rapid increase” in hemoglobin level means an increase in Hb level of at least about 1 g/dL in 4 weeks, preferably 1 g/dL in 2 weeks or 2 g/dL in about 4 weeks.

Methods of Identifying Inhibitors of HIF PH 1, HIF PH 2, HIF PH 3 and/or P4H-TM

The present invention provides methods of screening and identifying agents that increase endogenous erythropoietin through inhibition of HIF PH 1, HIF PH 2, HIF PH 3 or combinations thereof, in addition to inhibiting P4H-TM. In some embodiments, the identified agents inhibit HIF PH 1, HIF PH 2 and HIF PH 3, i.e., pan HIF PH 1-3 inhibitors, in addition to inhibiting P4H-TM. In other embodiments, the identified agents inhibit HIF PH 1 and HIF PH 3; HIF PH 1 and HIF PH2; or HIF PH 2 and HIF PH3; in addition to inhibiting P4H-TM. In further embodiments, the identified agents inhibit HIF PH 1, HIF PH 2, HIF PH 3 and P4H-TM enzymatic activity. In other embodiments, the identified agents inhibit P4H-TM activity without substantially inhibiting HIF PH activity.

Screening assays to identify agents that inhibit HIF PH 1, HIF PH 2, HIF PH 3 and/or P4H-TM enzymatic activity typically involve the monitoring of the consumption of a reaction substrate or the production of a reaction product. Isolation of a reaction product may be facilitated by a label such as biotin or a histidine tag that allows purification from other reaction components via precipitation or affinity chromatography. Detection of a reaction substrate or reaction product can involve fluorophores, radioactive isotopes, enzyme conjugates, and other detectable labels that are well known in the art. The results may be qualitative or quantitative.

One example of an inhibitory assay involves measuring the production of hydroxylated proline or asparagine residues in HIFα or a fragment thereof. In another example, the inhibitory assay involves measuring the formation of succinate from 2-oxoglutarate in the presence of cell lysate or purified enzyme and HIFα or a fragment thereof (See, e.g., Palmerini et al. J Chromatogr. 1985; 339:285-292; Cunliffe et al. Biochem J. 1986; 240:617-619.) An exemplary procedure that measures production of succinate from 2-oxoglutarate is described by Kaule and Gunzler. (Anal Biochem. 1990; 184:291-297.) Measuring and comparing enzyme activity in the absence and presence of a test agent identifies agents that inhibit hydroxylation of all HIF PH isozymes (HIF PH 1, HIF PH 2 and HIF PH 3), inhibit hydroxylation of one or more specific HIF PH isozymes, i.e., HIF PH 1 or inhibit hydroxylation of P4H-TM, depending on the experimental setup.

### Pharmaceutical Formulations and Routes of Administration

The compositions and compounds suitable for use in the method, or for manufacture of a medicament, of the present invention can be delivered directly or in pharmaceutical compositions containing excipients, as is well known in the art.

An effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, 17th ed., Mack Publishing Co.; and Hardman, Limbird, and Gilman, eds. (2001) The Pharmacological Basis of Therapeutics, 10th ed., McGraw-Hill Co.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred. In preferred embodiments, for use in the method of the invention the compounds of the present invention are administered orally.

Pharmaceutical dosage forms of a suitable compound for use in the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP; Inactive Ingredient Search for Approved Drug Products available through the U.S. Food and Drug Administration's website, and Handbook of Pharmaceutical Additives, ed. Ash; Synapse Information Resources, Inc. 2002.)

Pharmaceutical dosage forms of a compound for use in the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions for use in the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. For example, a dose can be calculated for animal models that should achieve a circulating concentration range of the administered agent or agents that approximate the IC₅₀ values that were determined in cell culture. Dosage ranges appropriate for human subjects can be determined, for example, using data obtained from animal studies and/or cell culture assays.

Dosages preferably fall within a range of circulating concentrations that includes the ED₅₀ with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In some embodiments, the therapeutically effective dosage of a HIF PH, HIF P4H-TM or HIF PH and PH4-TM inhibitor ranges from about 0.1 mg to about 10,000 mg, from about 1 mg to about 5000 mg, from about 5 mg to about 1000 mg, from about 10 mg to about 500 mg, from about 100 mg to about 500 mg, from about 100 mg to about 1000 mg, from about 100 mg to about 5000 mg or from about 500 mg to about 5000 mg, or any range in between.

In further embodiments, the therapeutically effective dosage of a HIF PH, HIF P4H-TM or HIF PH and PH4-TM inhibitor is at least about 0.1 mg, 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 750 mg, 1,000 mg, 2,500 mg, 5,000 mg or 10,000 mg. In other embodiments, the therapeutically effective dosage of a HIF PH or HIF P4H-TM inhibitor is not more than about 0.5 mg, 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 750 mg, 1,000 mg, 2,500 mg, 5,000 mg or 10,000 mg.

In additional embodiments, the combined therapeutically effective dosage of one or more HIF PH and one or more HIF P4H-TM inhibitor ranges from about 0.1 mg to about 10,000 mg, from about 1 mg to about 5000 mg, from about 5 mg to about 1000 mg, from about 10 mg to about 500 mg, from about 100 mg to about 500 mg, from about 100 mg to about 1000 mg, from about 100 mg to about 5000 mg or from about 500 mg to about 5000 mg, or any range in between.

As used herein, “substantially the same” is defined to mean that the values being compared are within a 5-fold, 10-fold or 20-fold difference from each other. For example, an agent that has an IC₅₀ of 0.2 μM is substantially the same as one that has an IC₅₀ of 1.0 μM.

In additional embodiments, effective treatment regimes for compounds of the invention include daily administration. Alternatively, the compounds of the invention can be administered once, twice or three times weekly.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Expression, Purification and Activity Assays of Recombinant HIF PHs and P4H-TM

To help understand P4H-TM's role in the regulation of EPO expression, recombinant P4H-TM was generated and compared to recombinant HIF PHs 1-3 in a prolyl hydroxylase inhibition assay. In brief, FLAG His-tagged HIF PHs 1-3 were expressed in H5 insect cells and purified with an anti-Flag M2 affinity gel (Sigma-Aldrich, Helsinki, Finland). (See Hirsila M. et al. FASEB J. 2005; 19(10):1308-1310) His-tagged P4H-TM lacking its transmembrane domain were expressed in Sf9 insect cells and purified with proBond resin (Invitrogen Corp., Carlsbad, Calif.). (See Koivunen P. et al. J Biol Chem. 2007; 282(42):30544-30552) The HIF PH 1, HIF PH 2 and HIF PH 3 activities were assayed by measuring the hydroxylation-coupled stoichiometric release of ¹⁴CO₂ from 2-oxo-[1-¹⁴C]glutarate with a synthetic peptide DLDLEMLAPYIPMDDDFQL (Innovagen AB, Lund, Sweden) corresponding to the C-terminal hydroxylation site in HIF-1α as a substrate (See Hirsila M et al. J Biol Chem. 2003; 278:30772-30780) The assays were performed in the presence of varying concentrations of a known HIF PH inhibitor (compound A, FibroGen Inc., San Francisco, Calif.). Since no synthetic substrate is available for P4H-TM, its prolyl hydroxylase inhibition assay was performed using the enzyme-catalyzed uncoupled decarboxylation of 2-oxo-[1-¹⁴C]glutarate without any peptide substrate. (Koivunem P. supra) BSA was omitted from the reaction mixtures.

The IC₅₀ values of compound A were determined for the purified enzymes by keeping the 2-oxoglutarate concentration constant (at 40 μM for HIF PHs 1-3 and 100 μM for P4H-TM) while increasing the concentration of compound A. The IC₅₀ obtained for HIF PHs 1-3 were about 0.2-0.3 μM. The IC₅₀ for P4H-TM was 40 μM.

To verify that the differences seen in IC₅₀ values were not due to the use of different assays, the IC₅₀ of compound A was also determined for HIF PH 2 using the uncoupled decarboxylation assay. An IC₅₀ of 0.2 μM was obtained demonstrating that the differences in IC₅₀ values of compound A for the HIF PH 1-3 enzymes (0.2-0.3 μM) and that of P4H-TM (40 μM) are not due to the use of different assays.

### Example 2

### Mouse Lines

To further elucidate P4H-TM's role in the regulation of EPO expression, a P4H-TM null mouse line (P4h-tm^{-/-}) and a double gene-modified HIF PH 2/P4H-TM mouse line (Hif-ph2^{gt/gt}/P4h-tm^{-/-}) were generated. It was hypothesized that if P4H-TM was involved in the regulation of EPO expression and erythropoiesis, then the P4H-TM null and double gene-modified HIF PH 2/P4H-TM mice would be more sensitive to a HIF prolyl hydroxylase inhibitor, resulting in larger increases in EPO expression, serum EPO levels, hemoglobin, hematocrit and reticulocytes compared to controls.

The P4H-TM null mice line was generated by targeting a LacZNeo cassette into exon 3 of the P4h-tm gene, leading to a truncated transcript of exons 1-2 and a split exon 3 fused to LacZNeo. As HIF PH 2 null mice die during embryonic development, a Hif-ph2 hypomorphic mice line (Hif-ph2^{gt/gt}) was used to produce the double gene-modified mouse line, Hif-ph2^{gt/gt}/P4h-tm^{-/-}. The Hif-ph2 hypomorphic mice line expresses lower amounts of wild-type Hif-ph2 mRNA in various tissues, about 35% of that in wild-type mice in the kidney and 85% in the liver (Hyvarinen J et al. J Biol Chem. 2010; 285(18):13646-13657). These mice do not have increased levels of EPO mRNA in kidney, increased serum concentrations of EPO, increased blood hemoglobin or increased hematocrit values. Both mouse lines were backcrossed to a C57BL/6 line. Since Hif-ph2^{gt/gt} mice produce low numbers of offspring, to obtain Hif-ph2^{gt/gt}/P4h-tm^{-/-} double gene-modified mice, Hif-ph2^{+/gt} mice were crossed with P4h-tm^{-/-} mice to produce Hif-ph2^{+/gt}/P4h-tm^{+/-} double heterozygous offspring. Crossings of Hif-ph2^{+/gt}/P4h-tm^{+/-} or Hif-ph2^{+/gt}/P4h-tm^{-/-} mice with Hif-ph2^{+/gt}/P4h-tm^{+/-} or Hif-ph2^{+/gt}/P4h-tm^{-/-} mice produced a few Hif-ph2^{gt/gt}/P4h-tm^{-/-} double homozygous mutants and littermates with the genotypes Hif-ph2^{+/+}/P4h-tm^{+/+}, Hif-ph2^{+/+}/P4h-tm^{+/-}, Hif-ph2^{+/gt}/P4h-tm^{+/+}, Hif-ph2^{+/gt}/P4h-tm^{+/-}, Hif-ph2^{+/gt}/P4h-tm^{-/-} and Hif-ph2^{gt/gt}/P4h-tm^{+/-}.

As the crossings of Hif-ph2^{+/gt}/P4h-tm^{+/-} or Hif-ph2^{+/gt}/P4h-tm^{-/-} mice with Hif-ph2^{+/gt}/P4h-tm^{+/-} or Hif-ph2^{+/gt}/P4h-tm^{-/-} mice produced only small numbers of female wild-type mice or none at all, it was decided to use not only wild-type, but also HifLph2^{+/+}/P4h-tm^{+/-}, Hif-ph2^{+/gt}/P4h-tm^{+/+} and Hif-ph2^{+/gt}/P4h-tm^{+/-} mice as littermate controls. This decision was based on previous data that indicated that there was no difference in hemoglobin and hematocrit values between homozygous Hif-p4h-2^{gt/gt} and wild-type mice. (Hyvarinen J et al. supra). Only female mice were used in all experiments.

### Example 3

### In Vivo Studies

Mice were orally administered compound A, 100 mg/kg, in a volume of 300 either once (acute exposure) or 3 times a week (on days 1, 3 and 5) for 3 or 5 weeks (chronic exposure). The drug was dissolved in 0.5% sodium carboxymethyl cellulose (NaCMC, Spectrum Chemicals, Gardena, Calif.) and 0.1% Polysorbate 80 (Sigma-Aldrich, Helsinki, Finland). The vehicle served as a negative control in the experiments. All the animal experiments were performed according to protocols approved by the Provincial State Office of Southern Finland.

### HIF Stabilization

To study whether the genetic deletion of P4H-TM stabilized HIF-1α or HIF-2α expression alone or in combination with a HIF PH inhibitor, Western blots were performed on kidney and liver samples obtained from P4h-tm^{-/-}, Hif-p4h-2^{gt/gt} and wild-type mice. In brief, snap-frozen tissue samples were crushed to a powder and lysed in 3 M urea supplemented with 25 mM Tris-HCl (pH 7.5), 75 mM NaCl and 0.25% Nonidet P-40. Following centrifugation, the supernatants were saved and the isolated nuclear fractions were homogenized in 10 mM Hepes (pH 7.9), 10 mM KCl, 1.5 mM MgCl₂, 0.1 mM EDTA, 0.1 mM EGTA, 0.1% Nonidet P-40 and 1 mM DTT supplemented with Complete EDTA-free (Roche Diagnostics Oy, Espoo, Finland). The homogenates were centrifuged and then the pellets were washed with PBS followed by resuspension in 20 mM Hepes (pH 7.9), 400 mM NaCl, 0.25 mM EGTA, 1.5 mM MgCl₂, 10% glycerol and 0.5 mM DTT. The mixtures were then incubated on a shaker at 4° C. for 20 min. The supernatant protein concentrations were determined by the Bradford method (Bio-Rad Protein Assay, Bio-Rad, Hercules, Calif.). The supernatants of the tissue homogenates and isolated nuclear fractions were resolved by SDS-PAGE and blotted onto Immobilon-P membranes (Millipore Oy, Espoo, Finland) that were blocked with Tris-buffered saline containing 5% non-fat dry milk. The membranes were then probed with the following primary antibodies: anti-HIF-1α (NB 100-479; Novus Biologicals, Littleton, Colo.), anti-HIF-2α (NB100-122; Novus Biologicals), anti-α-tubulin (α-tub) (B-6199; Sigma Aldrich, St. Louis, Mo.) and anti-β-actin (NB600-501; Novus Biologicals). Bound antibodies were detected with horseradish peroxidase-conjugated secondary antibodies (Dako Finland Oy, Helsinki, Finland) and ECL detection reagents (Thermo Fisher Scientific, Waltham, Mass.).

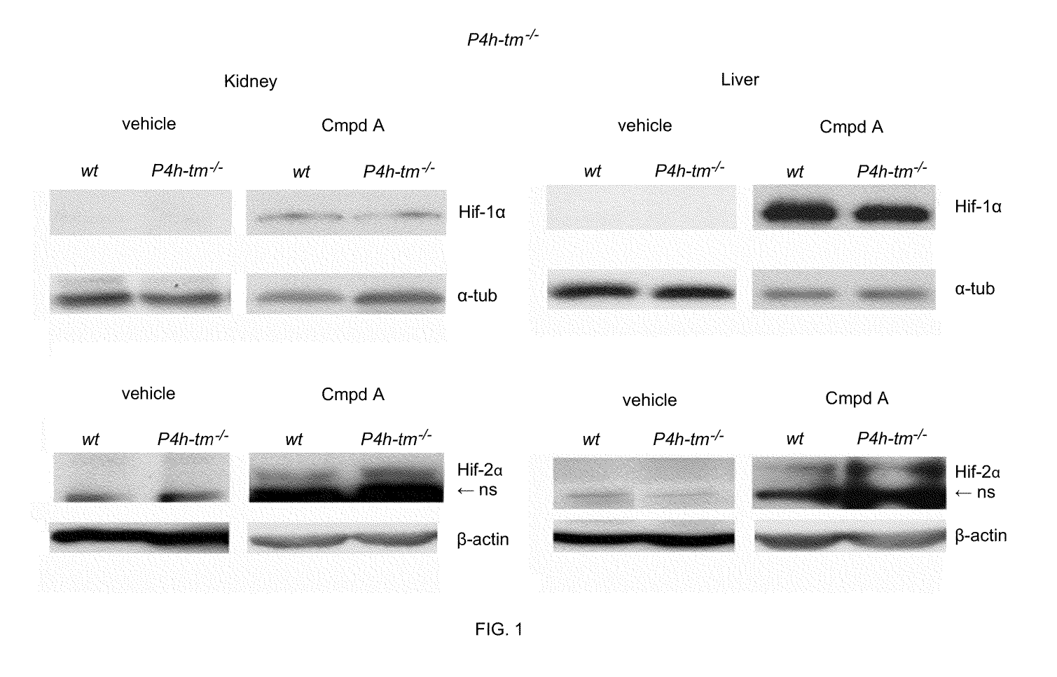

The results demonstrate that HIF-1α and HIF-2α are not stabilized in the kidney or liver of vehicle treated P4h-tm^{-/-} mice. (FIG. 1) Treatment with a HIF prolyl hydroxylase inhibitor, compound A, stabilized HIF-1α and HIF-2α in both tissues, the extent of this stabilization in the kidney being stronger in the P4h-tm^{-/-} than in the wild-type mice for Hif-2α, but not for Hif-1α. No differences in HIF-1α stabilization were seen in the liver between wild-type mice and compound A treated mice.

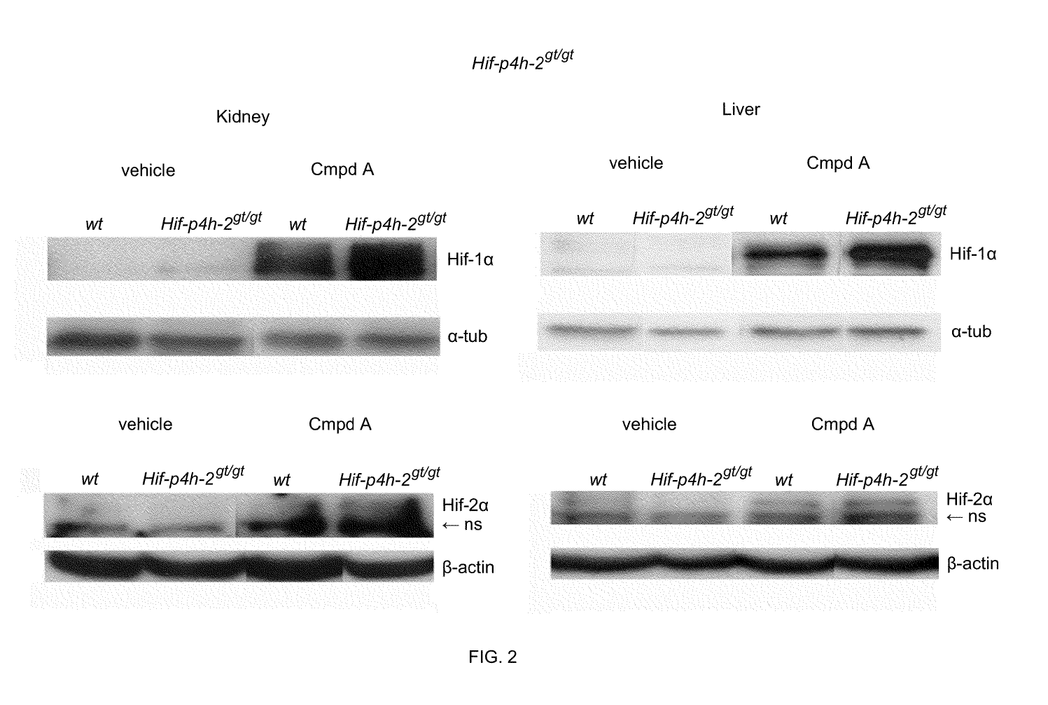

A slight degree of HIF-1α stabilization was seen in the kidney of Hif-p4h-2^{gt/gt} mice without compound A treatment. (FIG. 2) Treatment with compound A for 3 weeks stabilized Hif-1α and Hif-2α in the kidney and liver of both wild-type and Hif-p4h-2^{gt/gt} mice, although Hif-p4h-2^{gt/gt} mice exhibited a greater degree of stabilization in both organs compared to the wild-type mice.

### EPO Expression and Erythropoiesis Analysis

The role of P4H-TM in regulating EPO production and erythropoiesis was examined in P4h-tm^{-/-} and Hif-p4h-2^{gt/gt} mice. It was hypothesized that if P4H-TM was involved in the regulation of EPO production and erythropoiesis, then P4h-tm^{-/-} and Hif-p4h-2^{gt/gt} mice would be more sensitive to a HIF prolyl hydroxylase inhibitor compared to wild-type mice. It was further hypothesized that P4h-tm^{-/-} and Hif-p4h-2^{gt/gt} mice treated with a HIF PH inhibitor would exhibit, compared to wild-type mice, increased EPO mRNA levels, increased serum EPO levels, increased hemoglobin concentrations, increased hematocrit levels, increased reticulocytes percentage and decreased hepcidin mRNA levels. To this end, P4h-tm^{-/-} and Hif-p4h-2^{gt/gt} mice were treated with compound A and EPO mRNA levels, serum EPO levels, hemoglobin concentration, hematocrit level, percentage of reticulocytes and hepcidin mRNA levels were determined with vehicle-treated gene-modified and wild-type mice serving as controls.

### Quantitative Real-Time RT-PCR (Q-PCR) Analysis

mRNA levels were determined using quantitative real-time reverse transcriptase polymerase chain reaction (Q-PCR). In brief, kidney or liver tissues were dissected immediately after sacrifice, snap-frozen in liquid nitrogen and stored at −70° C. Total RNA was isolated using the TriPure Isolation Reagent (Roche Applied Science, Indianapolis, Ind.), and then further purified with the EZNA Total RNA Kit (OMEGA Bio-tek, Inc. Norcross, Ga.). Reverse transcription was then performed with the iScript cDNA Synthesis Kit (Bio-Rad). Q-PCR was performed with iTaq SYBR Green Supermix with ROX (Bio-Rad) and the Stratagene MX3005 thermocycler (Agilent Technologies, Santa Clara, Calif.). The sequences of the Q-PCR primers were: 5′-CATCTGCGACAGTCGAGTTCTG-3′ (SEQ ID NO:1) and 5′-CACAACCCATCGTGACATTTTC-3′ (SEQ ID NO:2) for EPO; 5′-GAATATAATCCCAAGCGATTTG-3′(SEQ ID NO:3) and 3′-CACACCATTTTTCCAGAACTG-5′ (SEQ ID NO:4) for TATA-binding protein (Tbp); 5′-AAGCAGGGCAGACATTGCGATACC-3′(SEQ ID NO:5) and 5′-AGATGCAGATGGGGAAGTTGGTGT-3′(SEQ ID NO:6) for Hepcidin; and 5′-CAATAGTGATGACCTGGCCGT-3′ (SEQ ID NO:7) and 5′-AGAGGGAAATCGTGCGTGAC-3′ (SEQ ID NO:8) for β-Actin. The expression data were normalized to Tbp or β-Actin.

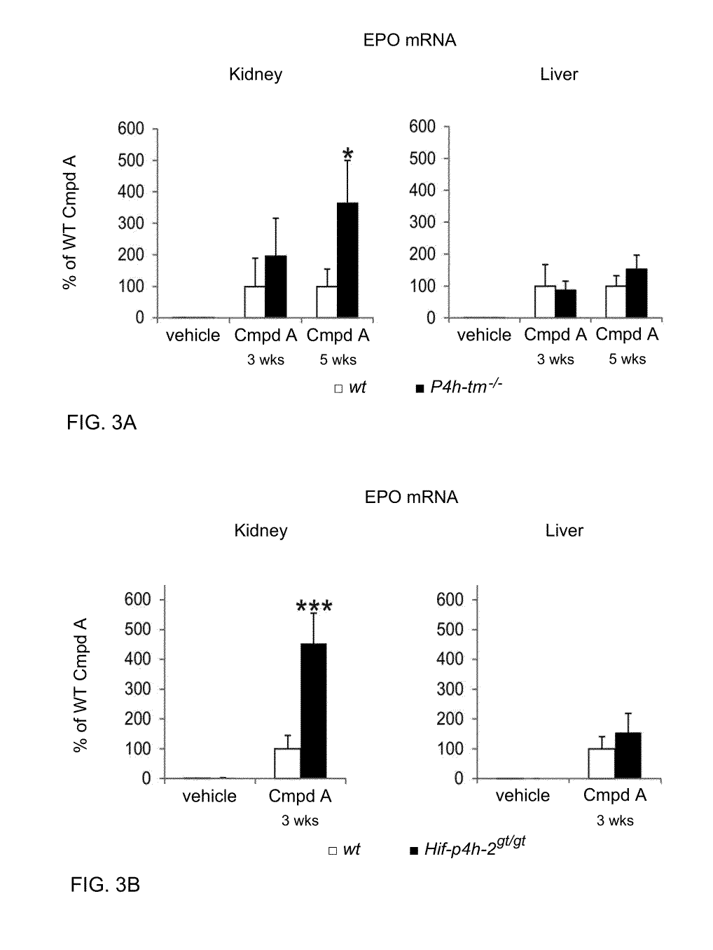

There were no significant differences in kidney or liver EPO mRNA expression for vehicle treated wild-type and P4h-tm^{-/-} mice. (FIG. 3A) Treatment with compound A significantly increased mean EPO mRNA level in the kidney about 2-fold at 3 weeks and 4-fold at 5 weeks in P4h-tm^{-/-} mice compared to wild type mice. (FIG. 3A) There was no significant difference in liver EPO mRNA expression between the two groups of mice. (FIG. 3A).

There were no significant differences in kidney or liver EPO mRNA expression for vehicle treated wild-type and HIF-ph2^{gt/gt} mice (FIG. 3B). Treatment with compound A significantly increased mean EPO mRNA level in the kidney about 4.5-fold at 3 weeks in Hif-p4h-2^{gt/gt}mice relative to the wild type. There was no significant difference in liver EPO mRNA expression between the two groups of mice. (FIG. 3B).

### Serum EPO Levels and Blood Cell Counts

Terminal blood samples were drawn from the inferior vena cava 6 h after the last administration of compound A. Blood cell counts were performed using a Cell-Dyn Sapphire (Abbott Laboratories, Abbott Park, Ill.). For serum EPO level determination, blood samples were allowed to clot overnight at 4° C. followed by centrifugation for 20 min at 1000 g. Serum EPO levels were determined using a Quantikine Mouse EPO Immunoassay kit (R&D Systems, Minneapolis, Minn.).

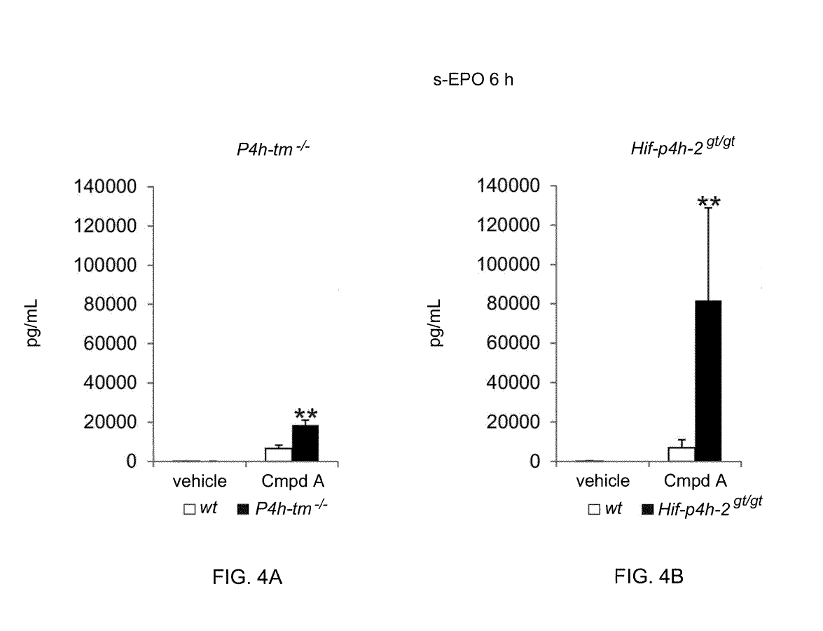

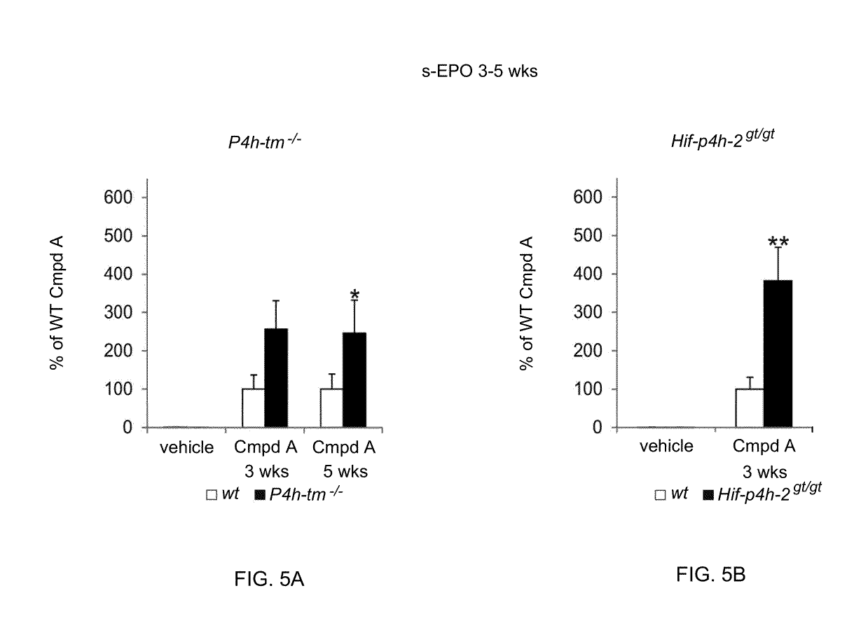

Serum EPO concentrations were equivalent between vehicle-treated wild-type and P4h-tm^{-/-} mice in the acute administration (6 h) experiment (FIG. 4A) and the chronic administration (3 or 5 weeks) experiment (FIG. 5A). Compound A administration markedly increased serum EPO concentration at all time points for wild-type and P4h-tm^{-/-} mice, but the increases were significantly elevated in the P4h-tm^{-/-} mice at 6 h and 5 weeks compared to the wild-type mice. (FIGS. 4A and 5A)

Similarly, serum EPO concentrations were equivalent between vehicle-treated wild-type and Hif-ph2^{gt/gt} mice in the acute administration (6 h) experiment (FIG. 4B) and the chronic administration (3 weeks) experiment (FIG. 5B). Compound A administration markedly increased serum EPO concentration at both time points for wild-type and Hif-ph2^{gt/gt} mice, but the increases were significantly elevated in the Hif-ph2^{gt/gt} mice compared to the wild-type mice. (FIGS. 4B and 5B)

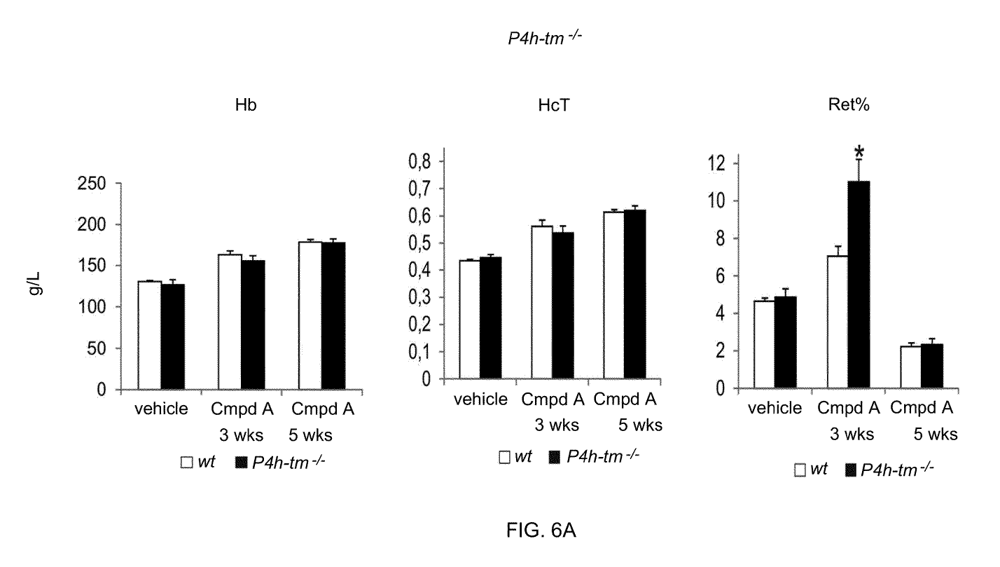

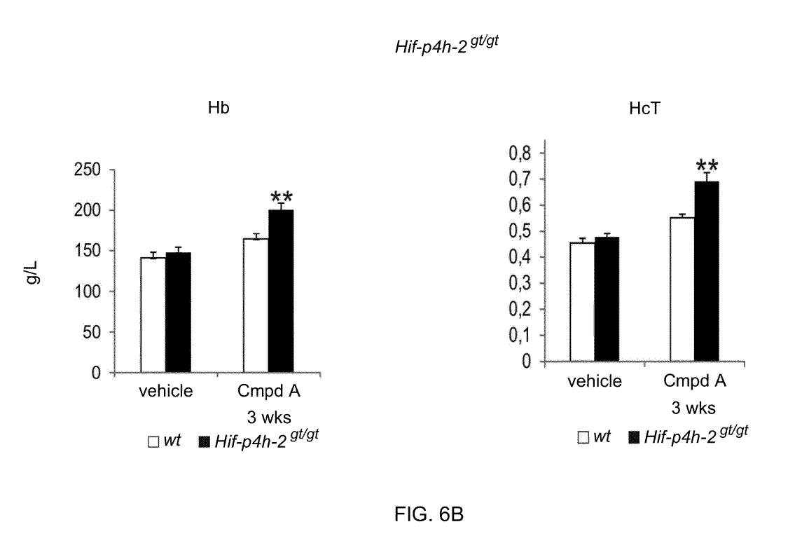

The hemoglobin content and hematocrit levels of vehicle-treated wild-type and P4h-tm^{-/-} mice were similar at each other. (FIG. 6A) Additionally, the hemoglobin and hematocrit levels of compound A-treated wild-type and P4h-tm^{-/-} mice were similar to each other. (FIG. 6A) The reticulocytes were significantly elevated at 3 weeks in compound A-treated P4h-tm^{-/-} mice treated with compared to compound A-treated wild-type mice, whereas there was no significant difference between the two groups of mice at 5 weeks. (FIG. 6A)

The hemoglobin content and hematocrit levels in vehicle-treated wild-type and Hif-ph2^{gt/gt} mice were similar. (FIG. 6B) In contrast, compound A treated Hif-ph2^{gt/gt} mice had significantly elevated hemoglobin and hematocrit levels compared to compound A treated wild-type mice. (FIG. 6B)

Although the serum EPO concentration had increased about 2.5-fold at 3 and 5 weeks and the reticulocyte counts had increased about 1.6-fold at 3 weeks in the compound A-treated P4h-tm^{-/-} mice as compared with the treated wild type, a similar increase was not seen in the hemoglobin and hematocrit values of the compound A-treated P4h-tm^{-/-} mice. This finding may be due to the fact that the serum EPO concentrations were already significantly elevated in the compound A-treated treated wild-type mice such that the further 2.5-fold increase in serum EPO concentrations of the compound A-treated P4h-tm^{-/-} mice may have been insufficient to further increase in the already elevated hemoglobin and hematocrit values.

### Hepcidin Expression

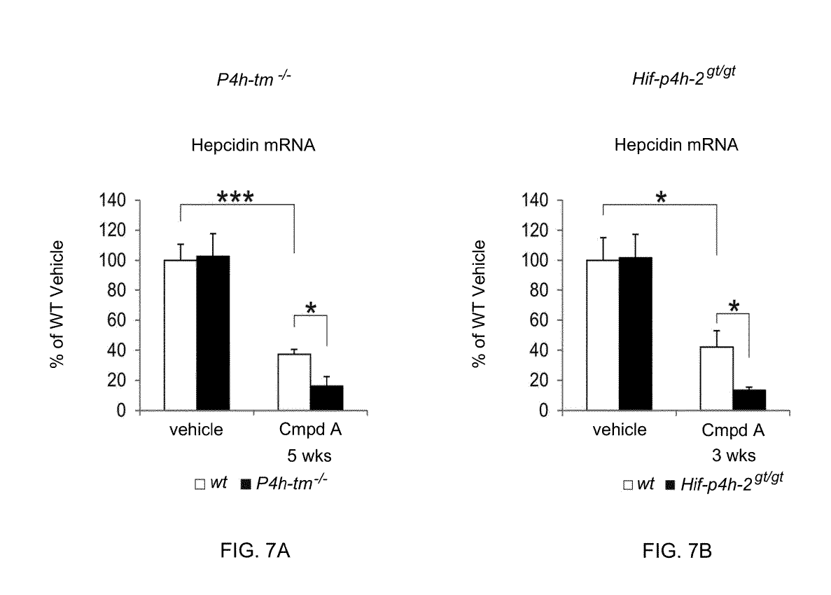

The effect of compound A administration on hepcidin mRNA expression was studied in wild-type, P4h-tm^{-/-} and Hif-ph2^{gt/gt} mice. Liver tissue was collected at the end of the chronic treatment periods and qPCR analysis performed. There was no significant difference in hepcidin mRNA levels between vehicle-treated wild-type and P4h-tm^{-/-} mice (FIG. 7A) or vehicle-treated wild-type and HIF-ph2^{gt/gt} mice (FIG. 7B). Compound A treatment lowered hepcidin mRNA level in wild-type mice by about 60-65%, but the magnitude of the decrease was even larger in the P4h-tm^{-/-} and HIF-ph2^{gt/gt} mice. (FIGS. 7A and 7B). In the case of compound A-treated P4h-tm^{-/-} mice, the hepcidin mRNA level was about 40% of that in the compound A-treated wild-type mice (FIG. 7A), while HIF-ph2^{gt/gt} mice was about 30% of that seen in the compound A treated wild-type mice (FIG. 7B).

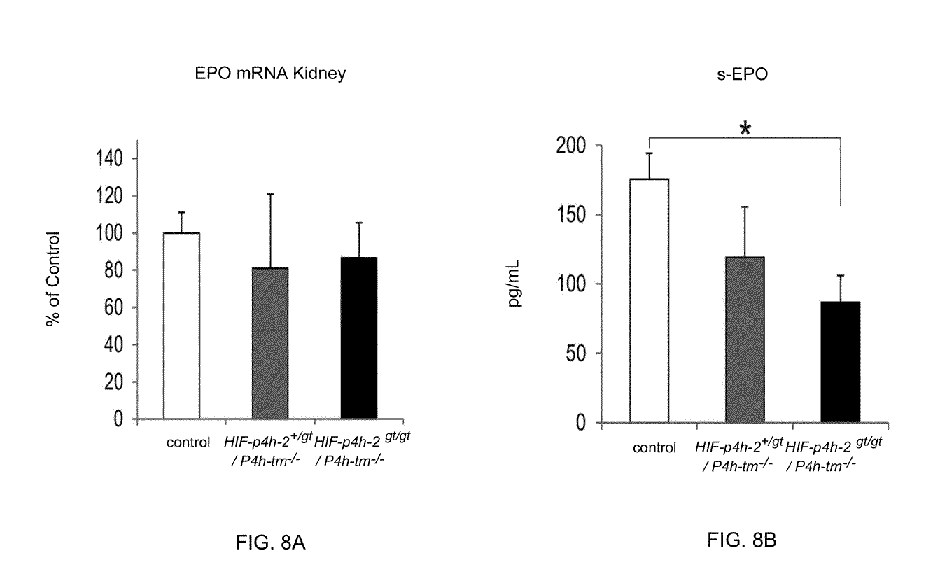

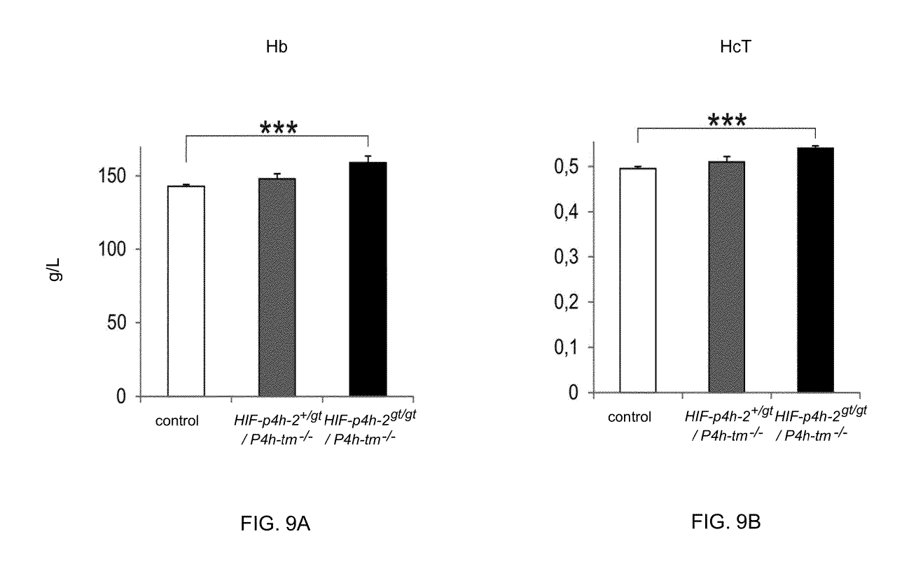

To further study the role of P4H-TM in erythropoiesis, the double gene-modified Hif-ph-2^{gt/gt}/P4h-tm^{-/-} mouse line was produce. These mice had an EPO mRNA level that was similar to controls, (FIG. 8A) but surprisingly, their mean serum EPO concentration was significantly lower than mean serum EPO concentration of the wild-type mice (FIG. 8B). The Hif-ph-2^{gt/gt}/P4h-tm^{-/-} mice also had mean hemoglobin concentration and hematocrit levels that were significantly higher than control mice (P<0.00005) (FIGS. 9A and 9B). The reasons for the decrease in serum EPO are unknown, but it has been speculated that an increased number of erythroid progenitors and early erythrocytes may have reduced free EPO protein molecules through increased receptor binding. It has also been reported that several patients with increased hemoglobin values due to heterozygous HIF-P4H-2 mutations have their serum EPO concentrations within the normal range, and in some cases even close to or at the lower limit of normal. These facts may explain why the Hif-ph-2^{gt/gt}/P4h-tm^{-/-} mouse with the lowest EPO concentration, 20.4 pg/mL had the highest hemoglobin value, 180 g/L.

### Example 4

### Identification of P4H-TM Inhibitors

A library of known HIF PH inhibitors is screened using the above assay that employs the P4H-TM-catalyzed uncoupled decarboxylation of 2-oxo-[1-¹⁴C]glutarate without the use of a peptide substrate. Five compounds are identified (compounds B-F) that have IC₅₀ values of about 0.2-0.5 μM for both HIF PH and P4H-TM activity. Compound B is selected for further study.

A chemical library devoid of known compounds that possess HIF PH inhibitory activity is screened for P4H-TM inhibitory activity using the P4H-TM-catalyzed uncoupled decarboxylation of 2-oxo-[1-¹⁴C]glutarate without the use of a peptide substrate. Three compounds, compounds G-I, are identified that have IC₅₀ values of about 0.2-1.0 μM. Compound G is selected for further study.

### Example 5

### In Vitro Increase in Erythropoietin Expression with an Agent that Inhibits HIF PH and P4H-TM Activity

Human cells derived from hepatocarcinoma (Hep3B) tissue (American Type Culture Collection, Manassas Va.) are seeded into 35 mm culture dishes and grown at 37° C., 20% O₂, 5% CO₂ in Minimal Essential Medium (MEM), Earle's balanced salt solution (Mediatech Inc., Herndon Va.), 2 mM L-glutamine, 0.1 mM non-essential amino acids, 1 mM sodium pyruvate, and 10% FBS. When cell layers reached confluence, the media is replaced with OPTI-MEM media (Invitrogen Life Technologies, Carlsbad Calif.) and cell layers are incubated for approximately 24 hours in 20% O₂, 5% CO₂ at 37° C. Compound B identified in the enzymatic screening assay above, compound A (positive control) or 1% DMSO (vehicle-negative control) are then added to existing media in individual wells and the cells incubated overnight.

Following incubation, the conditioned media is collected from cell cultures and analyzed for erythropoietin expression using a QUANTIKINE immunoassay (R&D Systems, Inc., Minneapolis Minn.) according to the manufacturer's instructions. An increase in expression of erythropoietin with compound B over that seen with compound A is observed confirming the hypothesis that a compound that significantly inhibits both HIF PH and HIF P4H-TM activity can induce a more robust EPO expression compared to a compound that has just HIF PH inhibitory activity.

### Example 6

### In Vitro Increase in Erythropoietin Expression with an Agent that Inhibits P4H-TM Activity

Compound G is assayed for its ability to increase in vitro expression of erythropoietin using the assay disclosed in Example 5 above. Compound A and 1% DMSO serve respectively as positive control and negative control. A moderate increase in expression of erythropoietin is observed with compound G.

### Example 7

### Treatment of Anemia Induced by Cisplatin

The ability of compound B to treat anemia associated with post-ischemic acute renal failure is assayed using a procedure described by Vaziri et al. (Am J Physiol. 1994; 266(3 Pt 2):F360-6.) Compound A serves as a positive control. In brief, twelve Sprague Dawley male rats (280-300 g) are obtained from Charles River Laboratories. On day 0, rats are treated by intraperitoneal injection with a single dose of saline (control; n=3) at 8 ml/kg, or cisplatin (CP; Bedford Laboratories, Bedford Ohio) at 10 mg/kg (10 ml/kg; n=9). Blood samples (0.2 ml) are collected on days 5, 9, and 16 as follows. Animals are anesthetized with isoflurane and 0.2 ml of blood is collected from the tail vein into a MICROTAINER EDTA-2K tube (Becton-Dickinson). Blood samples are processed for hematocrit to determine the degree of anemia produced in each animal.

Beginning on day 19, three cisplatin-treated rats and the untreated control group are administered by oral gavage once per day for 5 consecutive days with a 2 ml/kg volume of 0.5% CMC (Sigma-Aldrich). Another three of the cisplatin-treated rats are treated by oral gavage once per day for five consecutive days with a 2 ml/kg volume of 2.5% compound A (25 mg/ml in 0.5% CMC), the compound A group. The final three cisplatin-treated rats are treated by oral gavage once per day for five consecutive days with a 2 ml/kg volume of 2.5% compound B (25 mg/ml in 0.5% CMC), the compound B group. Blood samples (0.5 ml) are collected immediately prior to treatment and 4 days after treatment initiation. Blood samples are analyzed for CBC and reticulocyte counts. On day 9 after initiation of oral treatment, blood samples (0.1 ml) are collected and processed for hematocrit.

On day 19, prior to the start of treatment, the cisplatin-treated rats have a mean hematocrit that is 22% of the controls. Treatment with compound A increases hematocrit of cisplatin-treated animals starting 4 days after initiating treatment and is significantly higher than the cisplatin-treated controls by day 9 post-treatment. Treatment with compound B increases hematocrit levels in cisplatin-treated rats on day 4 post-treatment and results on day 9 post-treatment in a two-fold increase in hematocrit over and above that achieved with compound A. This increase in hematocrit in the compound B group over that seen in the compound A group demonstrates that the use of an inhibitor, such as compound B, that possess both HIF PH and P4H-TM inhibitory activity generates a more robust induction of hematocrit than a HIF PH inhibitor.

### Example 8

### Erythropoietin Production Following Bilateral Nephrectomy

The ability of an inhibitor of P4H-TM in combination with a HIF PH inhibitor to induce endogenous erythropoietin production in the absence of functioning kidneys is assayed using a procedure described by Jacobson et al. (Nature. 1957; 179:633-634.) Briefly, rats are anesthetized under isoflurane and a midline abdominal incision is made under sterile conditions. The kidney capsules are peeled off, the pedicles are ligated, and both kidneys are removed. The abdomen is then closed and the animals are allowed to recover.

The rats are then treated by oral gavage at 2 and 20 hours post-surgery with 0.5% carboxymethyl cellulose (vehicle-treated negative control, CMC; Sigma-Aldrich, St. Louis Mo.), compound A (100 mg/kg, positive control) or compound A at 50 mg/kg in combination with compound G, a P4H-TM inhibitor (50 mg/kg) identified in Example 4. (Compound A and compound G have substantially the same therapeutic dosing range based on IC₅₀ values obtained in cell culture experiments.) Blood samples (0.6 ml) are collected at 24 hours as follows. Animals are anesthetized with isoflurane and blood is collected from the tail vein into a MICROTAINER EDTA-2K tube (Becton-Dickinson). Blood samples are processed for erythropoietin levels, hematocrit, hemoglobin level and reticulocyte number.

Serum EPO levels are noticeably increased in bilaterally nephrectomized rats treated with compound A (100 mg/kg) relative to vehicle-treated bilaterally nephrectomized rats. The serum EPO levels for bilaterally nephrectomised rats treated with compound A (50 mg/kg) and compound G (50 mg/kg) are still further increased over the EPO levels seen with in the bilaterally nephrectomised rats treated with compound A at 100 mg/kg, demonstrating that a more robust induction of EPO can be achieved by combining the administration of an agent that inhibits P4H-TM activity with an agent that inhibits HIF PH activity.

### Statistical Analysis

The statistical analyses were performed using Student's two-tailed t test. Since the serum EPO and kidney and liver EPO mRNA values in the 3 week experiments were not distributed normally, the statistical analyses in these cases were performed after logarithmic transformation of the values.

What is claimed is:

1. A method for treating anemia in a subject in need thereof, the method comprising administering to the subject an effective amount of a first agent that inhibits HIF PH activity and an effective amount of a second agent that inhibits P4H-TM activity, thereby treating the anemia.

2. The method of claim 1, wherein the first and second agent are the same.

3. The method of claim 1, wherein HIF PH is selected from the group consisting of HIF PH 1, HIF PH 2, and HIF PH 3.

4. The method of claim 3, wherein the first agent inhibits HIF PH 1, HIF PH 2 and HIF PH 3 activity.

5. The method of claim 3, wherein the first agent inhibits HIF PH1 and HIF PH3 activity.

6. The method of claim 5, wherein the therapeutic dosing ranges for the first agent to inhibit HIF PH1 and HIF PH 3 activity are substantially the same.

7. The method of claim 3, wherein the first agent inhibits HIF PH2 and HIF PH3 activity.

8. The method of claim 7, wherein the therapeutic dosing ranges for the first agent to inhibit HIF PH2 and HIF PH 3 activity are substantially the same.

9. A method for treating anemia in a subject in need thereof, the method comprising administering to the subject an effective amount of an agent that inhibits P4H-TM activity, thereby treating the anemia.

10. A method for increasing endogenous erythropoietin in a subject in need thereof, the method comprising administering to the subject an effective amount of a first agent that inhibits HIF PH activity and an effective amount of a second agent that inhibits P4H-TM activity, thereby increasing endogenous erythropoietin.

11. A method for increasing hematocrit in a subject in need thereof, the method comprising administering to the subject an effective amount of a first agent that inhibits HIF PH activity and an effective amount of a second agent that inhibits P4H-TM activity, thereby increasing hematocrit.

12. A method for increasing hemoglobin in a subject in need thereof, the method comprising administering to the subject an effective amount of a first agent that inhibits HIF PH activity and an effective amount of a second agent that inhibits P4H-TM activity, thereby increasing hemoglobin.

13. A method for increasing reticulocytes in a subject in need thereof, the method comprising administering to the subject an effective amount of a first agent that inhibits HIF PH activity and an effective amount of a second agent that inhibits P4H-TM activity, thereby increasing reticulocytes.

14. A method for increasing erythrocytes in a subject in need thereof, the method comprising administering to the subject an effective amount of a first agent that inhibits HIF PH activity and an effective amount of a second agent that inhibits P4H-TM activity, thereby increasing erythrocytes.

15. A method for decreasing hepcidin in a subject in need thereof, the method comprising administering to the subject an effective amount of a first agent that inhibits HIF PH activity and an effective amount of a second agent that inhibits P4H-TM activity, thereby decreasing hepcidin.
